(19) United States
(12) Patent Application Publication
(10) Pub. No.: US 2006/0241437 A1
(43) Pub. Date: Oct. 26, 2006

(54) Pachymeter

(75) Inventors: Scott Phillips, Victoria (CA); Bjarne Hansen, Victoria (CA); Timothy Ehrecke, Bettendorf, IA (US)

Correspondence Address: KLARQUIST SPARKMAN, LLP, 121 SW SALMON STREET, SUITE 1600, PORTLAND, OR, 97204, US

(73) Assignee: Portable Ophthalmic Devices, Inc., Bettendorf, IA (US)

(21) Appl. No.: 10/552,849

(22) PCT Filed: Apr. 8, 2004
(86) PCT No.: PCT/CA04/00537

§ 371 Date: Oct. 11, 2005

Related U.S. Application Data
(60) Provisional application No. 60/461,833, filed on Apr. 11, 2003.

(51) Int. Cl. A61B 8/00

(52) U.S. Cl. 600/438; 600/437

(57) ABSTRACT

A pachymeter apparatus for measuring corneal thickness is configured for the calculation of intraocular pressure (“IOP”). The apparatus includes a pachymeter for measuring corneal thickness as well as a microprocessor and a user interface. A reading of IOP from a tonometer can be entered into the device and corrected according to the pachymeter reading of corneal thickness. Correction algorithms are user selectable and user customizable. A pachymeter may have a graphical display which displays multiple locations at which measurements are to be taken. A pachymeter may use a capacitor charged by a variable current supply to measure times for signals to be reflected from a back side of a cornea.

### CROSS REFERENCE TO RELATED APPLICATION

This application claims the benefit of U.S. application No. 60/461,833 filed on 11 Apr. 2003 and entitled PACHYMETER WITH PRESSURE CORRECTION FEATURE.

### BACKGROUND OF THE INVENTION

This invention relates to a convenient apparatus for the calculation of corrected intraocular pressure (IOP) according to variations in corneal thickness.

A corneal pachymeter can be used to measure the thickness of the cornea. The basic structure of a typical ultrasonic corneal pachymeter is relatively simple. An ultrasonic pulse is created by a pulse generating circuit and converted to ultrasonic vibrations by a transducer which sends ultrasonic waves into a cornea. The ultrasonic waves reflect from the back of the cornea to create a reflected signal. The time elapsing between the generation of the ultrasonic vibrations and detection of the reflected signal is measured. Since the speed of sound in corneal tissue is fairly well known, the thickness of the cornea can be determined from the measured time.

Typically pachymeters have been used to provide information about corneal thickness in refractive surgery. Recently however the need for pachymeters in the diagnosis and management of glaucoma has become apparent.

Glaucoma is a group of diseases characterized by optic nerve damage and is highly correlated with high IOP. Primary open angle glaucoma (POAG) is a leading cause of blindness and currently affects roughly 2.2 million Americans, or roughly 2% of the population 40 and older.

IOP is typically estimated by using an tonometer such as the Goldmann applanation tonometer. Goldman tonometers work by measuring the force required to compress the cornea until a round area of a certain diameter has been flattened. The required force depends upon various factors including intraocular pressure, corneal stiffness and other factors such as scleral elasticity and the stiffness and geometry of other ocular anatomical structures. Typical tonometers provide outputs measured in mmHg.

At present common practice is to use tonometry alone to measure IOP. However some research has indicated that corneal thickness has a significant impact on IOP measurement. For example Orssengo et al., Ehlers et al., Doughty et al., and Whitacre et al. all attempt to determine an appropriate formula to link tonometry and IOP.

In 2002 a very large multicenter American study of IOP and glaucoma called the Ocular Hypertension Study (OHTS) was published. One of the conclusions was a recommendation to measure central corneal thickness in all patients with ocular hypertension or glaucoma.

There is still significant uncertainty in the field as to which correction formula to use. The OHTS study group did not use any of the available correction formulas since they felt that they were either theoretical in nature or else were based on too small and ethnically homogeneous a sample group to be trustworthy.

There is a need for convenient apparatus and methods for measuring corneal thickness. There is a need for methods for measuring IOP which are more accurate than tonometry alone.

### SUMMARY

This invention has various aspects. One aspect of the invention provides a device for determining corrected intraocular pressure. The device comprises a corneal pachymeter; a microprocessor which automatically receives corneal thickness data from the pachymeter; and an input device for allowing a tonometer reading from a separate tonometer to be entered. The microprocessor is configured to perform at least one algorithm for modifying the tonometer reading based on the corneal thickness data to produce a corrected intraocular pressure value. The device includes a display for displaying the corrected intraocular pressure value.

In some embodiments of the invention the pachymeter comprises a pulse generator; an ultrasonic transducer connected to generate an ultrasonic pulse in response to a pulse from the pulse generator; and, a circuit for amplifying ultrasonic signals reflected at a back side of the cornea, detecting these reflected signals and timing them.

Another aspect of the invention provides a pachymeter comprising a circuit for amplifying signals reflected from the cornea, detecting the reflected signals and timing them. The circuit comprises a timing circuit comprising: a capacitor; a charging circuit connected to charge the capacitor at a rate which decreases with time; a trigger circuit connected to cause the charging circuit to commence charging the capacitor from an initial state of charge when the pulse is delivered by the pulse generator; and, a digital to analog converter connected to measure a voltage across the capacitor as of a time at which the reflected signals are detected.

Another aspect of the invention provides a pachymeter comprising a display and at least one stored set of a plurality of predetermined locations for taking a set of pachymeter measurements. The display is configured to display a graphical representation of an eye marked with indicia to graphically indicate one of the predetermined locations at which a next pachymeter measurement of the set ought to be taken. In some embodiments, upon obtaining the next pachymeter measurement the display is automatically configured to cause the indicia to graphically indicate another one of the predetermined locations.

Another aspect of the invention provides a device for measuring thickness of a layer. The device comprises: a transmitting transducer connected to transmit an ultrasonic pulse into a layer to be measured; a receiving transducer connected to detect the ultrasonic pulse after the ultrasonic pulse has been reflected from a back side of the layer; and a timing circuit. The timing circuit comprises a capacitor; a charging circuit connected to charge the capacitor at a rate which decreases with time; a trigger circuit connected to cause the charging circuit to commence charging the capacitor from an initial state of charge when an ultrasonic pulse is transmitted into the layer by the ultrasonic transducer; and a digital to analog converter connected to measure a voltage across the capacitor as of a time at which the reflected ultrasonic pulse is detected at the receiving transducer.

These aspects of the invention may be provided individually or in any combination with one another.

Other aspects of the invention provide methods for obtaining managing and storing information about the thickness of subjects' corneas.

Further aspects of the invention and features of embodiments of the invention are set out below.

### BRIEF DESCRIPTION OF THE DRAWINGS

In drawings which illustrate non-limiting embodiments of the invention:

FIG. 1 is a block diagram of a pachymeter according to an embodiment of the invention;

FIG. 2 is a block diagram of a pachymeter according to another embodiment of the invention;

FIG. 3 is a diagram illustrating a time measurement circuit in a pachymeter according to some embodiments of the invention;

FIGS. 4A and 4B show portions of a user interface of a pachymeter according to embodiments of the invention.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense. The schematic elements illustrated in the drawings are indicative of functions and not necessarily separate components.

A pachymeter according to this invention may be used by clinicians to measure corneal thickness. Corneal thickness information may be used to adjust readings (which may be obtained from existing tonometers) to yield corrected IOP values. Some embodiments of the invention provide pachymeters having mechanisms which permit clinicians to select and/or adjust the correction formulas used to provide corrected IOP values.

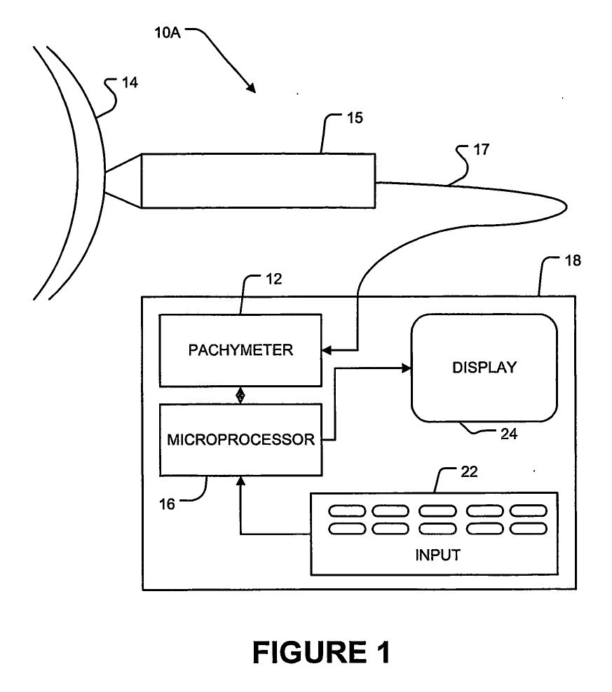

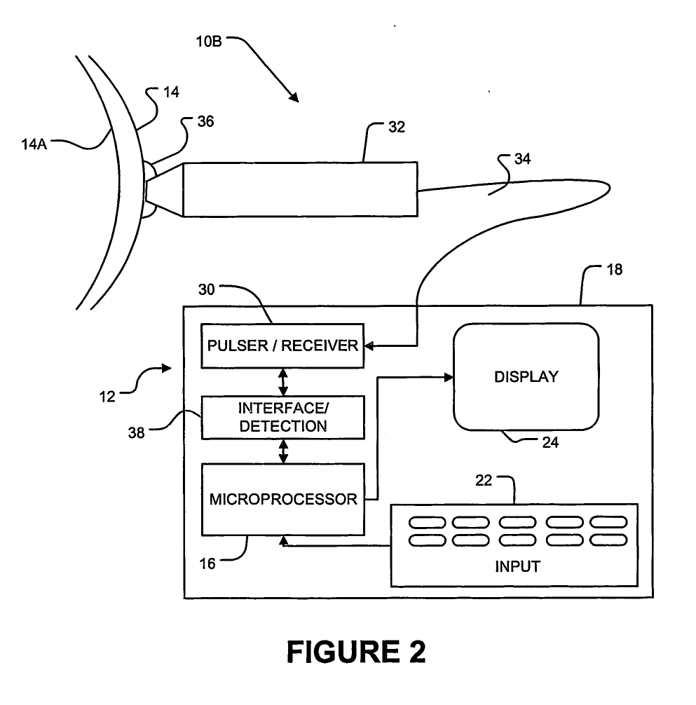

FIG. 1 shows an apparatus 10A according to one embodiment of the invention. Apparatus 10A includes a pachymeter 12. Pachymeter 12 can measure the thickness of cornea 14. Pachymeter 12 comprises any suitable device for measuring corneal thickness which produces an output signal (a “thickness signal”) indicative of a measured corneal thickness. Various suitable types of pachymeter are known in the field. In some embodiments of the invention, pachymeter 12 comprises an ultrasonic pachymeter. FIG. 2 is an example of an ultrasonic pachymeter according to the invention.

Apparatus 10A may be integrated into a housing 18 which may be small and handheld. Housing 18 may have a form factor similar to that of a personal digital assistant (“PDA”) such as a PALM™ PDA.

In the illustrated embodiment of the invention, pachymeter 12 comprises a probe 15 which generates signals indicative of corneal thickness. The signals are transmitted to pachymeter 12 via a cable 17 or wireless communication, for example. Pachymeter 12 may or may not be in the same housing 18 as other components of apparatus 10A. Pachymeter probe 15 may be detachable and may plug into housing 18.

Pachymeter 12 generates the thickness signal and makes the thickness signal available to a microprocessor 16. Microprocessor 16 executes software instructions which control the overall operation of apparatus 10A and provide one or more formulas for allowing a corrected IOP to be determined from an uncorrected IOP measured by a tonometer and a corneal thickness measurement. The software instructions may be stored in a suitable memory internal to microprocessor 16 or in a suitable program memory accessible by microprocessor 16.

Apparatus 10A includes a user interface which includes an input device 22 and a display 24. The display 24 and input device 22 may be provided by a touchscreen incorporated into housing 18. A clinician can enter a tonometer reading (i.e. an IOP value measured by any suitable tonometer) into apparatus 10A by way of input device 22. Input device 22 may comprise a keyboard, a key pad, a touch screen, a trackball, a pen-based input system, a combination of such input systems or any other suitable and equivalently useful input device such as a voice recognition input.

To use apparatus 10A a clinician obtains a tonometer reading for a subject's eye. Any suitable tonometer, of which many are known in the field, may be used to obtain this reading. The clinician then enters the tonometer reading into apparatus 10A by way of input device 22.

The clinician also operates pachymeter 12 to acquire one or more measurements of corneal thickness for the subject's eye. The pachymeter readings may be obtained before or after entering the tonometer reading into apparatus 10A. In some embodiments of the invention, a measurement is made by touching transducer 15 to the cornea 14 of a subject with pachymeter 12 turned on. Pachymeter 12 will typically have features to determine when transducer 15 is in the correct position to detect the thickness of cornea 14. Such features are known in the pachymeter art. Multiple readings may be taken either automatically or under manual control to allow data averaging and variability analysis. In one embodiment of the invention, pachymeter 12 automatically makes a number (for example 4 to 12) of corneal thickness measurements each time transducer 15 is touched to the cornea 14 of a subject with pachymeter 12 turned on.

Microprocessor 16 applies the selected correction function to determine a corrected IOP. The correction function may use as inputs the tonometer reading and the corneal thickness measurement(s). In some embodiments the correction function involves computing a linear combination of the corneal thickness and one or more tonometer reading values. The corrected IOP is then displayed on display 24 either automatically or as a result of input from the operator.

The IOP correction formula may be a simple linear approximation. Data from one paper in the field for example indicates a zero correction point at about 520 microns corneal thickness with about 0.7 mmHg in correction for each 30 microns of corneal thickness change. This relationship can be expressed in the form DP=mT+b where DP is the pressure correction due to cornea thickness and T is the measured corneal thickness with m and b given approximately by m=0.023 and b=−12.13. A clinician may wish to adjust either of the two variables m and b to customize the zero point or the sensitivity of the formula.

Where such a formula is used, apparatus 10A may output the value for the correction DP, a corrected value for IOP, or both. Embodiments of the invention which produce as outputs a value for DP from correction formulae which do not require a tonometer reading as an input do not need to receive a tonometer reading.

Some embodiments of apparatus 10A provide a plurality of different correction functions. In a method of using such embodiments of the invention, a clinician selects one of a plurality of correction functions using input 22. In such case, apparatus 10A preferably includes a display (which could be display 24) which indicates which of the correction functions is currently selected. Apparatus 10A preferably permits a selected correction function to be saved so that the selected conversion function will remain selected, even if apparatus 10A is powered off, until a different correction function is selected.

Alternative formulae for determining the correction DP, a corrected value for IOP, or both may be provided instead of, or in addition to, the simple linear formula given above. Such alternative formulae may use linear regression or polynomial curve fits or multiple linear segments, for example. Such formulae may take as an input just corneal thickness for example as in the above formula or might include a tonometer reading as an input as well.

Microprocessor 16 may implement computing a corrected IOP value based on a selected formula in any of various ways. In some embodiments of the invention the formula comprises a lookup table. In such embodiments the lookup table may have, for example, one or two dimensions. The table might either generate a DP value as a function of cornea thickness T or a DP value based on both T and P or possibly generate an adjusted pressure value based on both T and P.

Some embodiments of apparatus 10A are configured to permit a clinician to set one or more parameters of one or more of the correction functions.

The corneal thickness used as an input by a correction function is a single measurement in some embodiments of the invention or a combination (such as an average) of two or more measurements in other embodiments of the invention. In some embodiments of the invention the operator can select the corneal thickness value(s) to be used in the calculation. In some embodiments the instrument selects the corneal thickness value(s) used in the calculation automatically.

FIG. 2 shows schematically an apparatus 10B according to a specific embodiment of the invention. Except as described below, apparatus 10B operates in a manner the same as or similar to apparatus 10A (apparatus 10A and apparatus 10B are referred to collectively as apparatus 10 herein). In apparatus 10B, pachymeter 12 uses the reflection of ultrasonic signals to determine corneal thickness.

Electric signals are generated by pulser/receiver 30 and transmitted to transducer 32 which generates ultrasonic vibrations which are transmitted into cornea 14. Transducer 32 may, for example, comprise a piezoelectric transducer. Transducer 32 is connected to pulser receiver 30 by a conductor 34. Conductor 34 may comprise a flexible coaxial cable, for example, so that transducer 32 can be moved separately from housing 18. In the alternative, transducer 32 may be mounted in a more solid manner to housing 18. Transducer 32 is in close proximity to or contact with cornea 14 and may be coupled acoustically using acoustic coupling fluid 36 which could be a saline solution, for example.

The ultrasonic vibrations generated by transducer 32 travel through cornea 14. At least some of the vibrations are reflected at the posterior surface 14A of cornea 14. The reflected vibrations travel back to the transducer 32 and are converted back into electric signals which are in turn amplified by pulser/receiver 32.

Pulser/receiver 30 comprises at least a pulse generator and a signal amplifier. These elements may be switched into the circuit in alternation so that the amplifier is not exposed directly to the pulse signal. From the amplifier, a detected signal is provided to interface detection circuit 38 which detects the pulse signal from the posterior surface 14A of cornea 14 and determines the time interval between the original pulse and the reflected pulse. The time delay signal is provided to microprocessor 16 (or a separate processor) which may use it to determine the thickness of cornea 14. Microprocessor 16 then performs the IOP correction. Microprocessor 16 may comprise a number of separate processors with functions divided between them in any suitable manner.

Determining the timing of a reflected pulse may involve detecting a peak of the reflected pulse. This may be performed by a peak detector circuit. The peak detector circuit function may be performed by microprocessor 16 if the signal from the pulser receiver 32 is digitized directly.

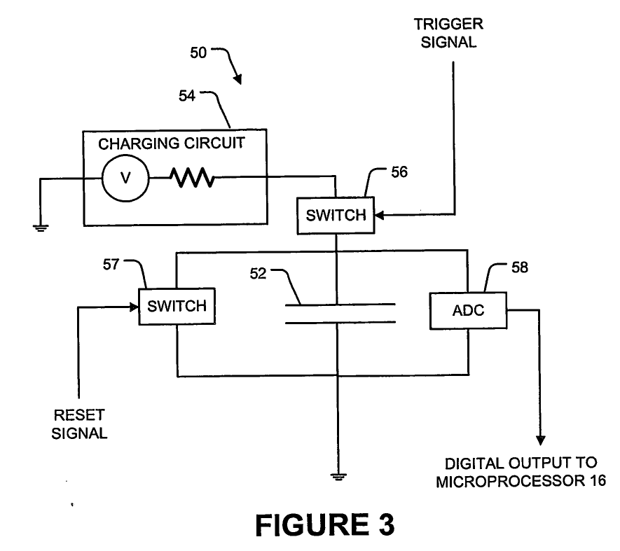

FIG. 3 illustrates a timing circuit 50 that is used in some embodiments of the invention to measure the time between the generation of an ultrasonic pulse at transducer 32 and the receipt of a reflected pulse. Timing circuit 50 comprises a capacitor 52, a charging circuit 54, an electronically controlled switch 56 and an analog to digital converter 58. Timing circuit 50 begins timing when it receives a START signal indicating that an ultrasonic pulse is being generated at transducer 32 and stops timing when it receives a STOP signal indicating that a reflected pulse has been detected. The STOP signal may be generated, for example, by a peak detector circuit.

Timing begins with capacitor 52 in a known state of charge (for example, discharged). At the same time as transducer 32 is caused to generate an ultrasonic pulse, the START signal causes switch 56 to turn on. This permits charging circuit 54 to begin charging capacitor 52. As capacitor 52 charges, the voltage across capacitor 52 increases. When a reflected pulse is detected, switch 56 is turned off. The voltage across capacitor 52 is then captured by digital to analog converter 58. The measured voltage across capacitor 52 is a function of the time elapsed between switch 56 opening and switch 56 closing. A switch 59 may be provided to discharge capacitor 52 after a measurement has been made.

Charging circuit 54 preferably charges capacitor 52 in a manner such that the rate at which voltage across capacitor 52 rises decreases with time. This behavior permits digital to analog converter 58 to measure voltages corresponding to short times with relatively high resolution while still being able to measure voltages corresponding to much longer times within its range. For example, charging circuit 54 may comprise a resistor 59 connected to a source of a constant voltage V₀. In this case, the voltage V(t) across capacitor 52 will increase with time, t, according to:

$$V ( t ) = {{V}_{0} ( 1 - e )}^{- \frac{t}{RC}} \qquad ( 1 )$$

where: C is the capacitance of capacitor 52; and R is the resistance of resistor 59. It can be seen from equation (1) that voltage increases relatively quickly at first. The rate at which voltage across capacitor 52 rises decreases with time. The voltage measured across capacitor 52 may easily be converted to corneal thickness in software calculations.

From Equation (1), the time resolution (Δt) is related to the voltage resolution (ΔV) of the analog to digital converter by
Δt=ΔV/K^{e}^{^{−Kt}} (2)
This is a well defined exponential function. It can be seen that the numerical resolution with which various time intervals can be measured is not constant. Measurements of short intervals (corresponding to thin corneas) can be made more precisely than measurements of longer intervals. This improvement in precision for short intervals can result in a reduction in the requirement for the number of resolution steps provided by the analog to digital converter.

In the alternative, the capacitor could be charged with a constant current source. In this case the capacitor voltage would change linearly with time according to:

$$V ( t ) = \frac{It}{C} \qquad ( 3 )$$

where I is the constant current, t is the time and C is the capacitance of the capacitor. Where Equation (3) applies, any time interval is linearly related to the numerical value resulting from the analog to digital conversion. This value can be multiplied by a constant related to sound speed in the cornea to yield a measure of the corneal thickness. This operation can be accomplished in subsequent software processing steps.

In embodiments in which Equation (3) applies, the resolution with which the thickness of the cornea can be measured is defined by the fixed step size of the resolution of the A/D converter used to measure the voltage across the capacitor, and is constant throughout the time measurement range. At any point the time resolution, Δt, is Δt=KΔV where ΔV is the required A/D converter resolution.

Facilities which provide some additional functions are desirable to ensure that apparatus 10 will work in the most effective manner. Interface detection circuit 38 may incorporate a facility to detect when the tip of transducer 32 is immersed in fluid by observing changes in signal strength. Such a facility can indicate to microprocessor 16 when a cornea may be present. The desired condition of perpendicularity of transducer 32 to the cornea 14 can be identified by, for example, monitoring signals from pulser/receiver 30 to determine when:

a strength of detected reflected signals is maximized,

a measured thickness is stable;

a measured thickness is minimized; or,

some combination thereof.

In some clinical settings it is desirable to use a pachymeter to obtain a set of measurements of the corneal thicknesses measured at different locations on a subject's eye. In such cases, keeping accurate track of the locations at which the individual measurements are made is important. Apparatus 10 may have a function for prompting a clinician to take a series of pachymeter measurements at predefined locations on a subject's eye. Apparatus 10 may store a number of sets of predefined locations.

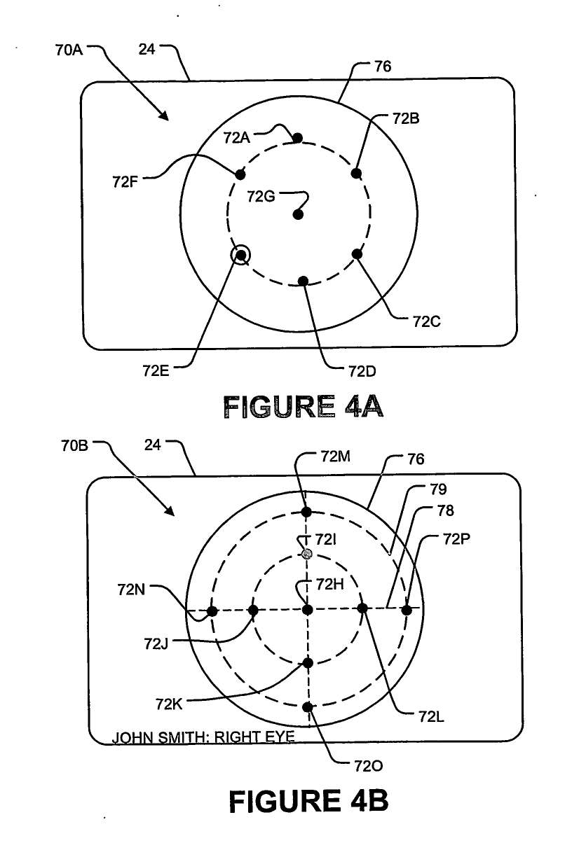

In some embodiments of the invention a user can cause apparatus 10 to commence the acquisition of such a series of measurements. In response, apparatus 10 displays on display 24 a schematic representation of an eye and an indicator which shows where on the eye the current measurement is to be taken. Example displays 70A and 70B are shown in FIGS. 4A and 4B respectively. Display 70A includes a circle 76 representing the subject's eye and spots 72A through 72G representing locations at which pachymeter measurements should be made. In display 70B spots spots 72H through 72P are located on two circles representing two different radii from the centre of the subject's eye and representing locations at which pachymeter measurements should be made. Spots 72A through 72G or 72H through 72P are collectively referred to herein as spots 72.

In the example embodiment shown in FIG. 4A, the next measurement should be made at a location 72E. Location 72E is highlighted, for example, by blinking, being identified by a selection icon, being enlarged, presented more darkly, being presented in a special color, or the like. Display 70 may include additional indicia, such as lines 78 or circles 79 to provide a clear indication of where each measurement is to be taken.

One or more pachymeter measurements are made at a location corresponding to each of spots 72 of the current set of measurements. Apparatus 10 stores each pachymeter reading and associates it with the location at which it is to be taken. The set of stored pachymeter readings can then be reviewed on display 24 or downloaded to another device for storage or further processing.

An apparatus 10 which displays graphical indicators as shown, for example, as shown in FIGS. 4A and 4B may be configured to perform a method which begins by allowing an operator to select a pattern of measurements to be made. In response to the selection apparatus 10 displays a graphical representation of the pattern on display 24 and highlights a spot indicating a location at which a first measurement should be made. In some embodiments apparatus 10 also displays the name of the intended subject and the eye (right or left) on which the measurements should be made.

The clinician makes the measurement. In some embodiments, apparatus 10 automatically makes a series of measurements at each location. Upon successful completion of the measurement(s) at each location, apparatus 10 automatically stores the measurements and associates them with the location (and preferably the name of the subject and the eye to which they pertain). Apparatus 10 then highlights a spot indicating the next location at which a measurement should be made. This process continues until measurements have been made at all locations in the selected pattern of measurements or the clinician terminates collection of measurements.

Certain implementations of the invention comprise computer processors which execute software instructions which cause the processors to perform a method of the invention. For example, a pachymeter may comprise a computer processor which executes software instructions which cause the processor to generate displays as shown in FIGS. 4A and 4B. The program product may comprise any medium which carries a set of computer-readable signals comprising instructions which, when executed by a computer processor, cause the data processor to execute a method of the invention. The program product may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like or transmission-type media such as digital or analog communication links.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

As will be apparent to those skilled in the art in the light of the foregoing disclosure, many alterations and modifications are possible in the practice of this invention without departing from the spirit or scope thereof.

1. A device for determining corrected intraocular pressure comprising:
a corneal pachymeter;
a microprocessor which automatically receives corneal thickness data from said pachymeter;
an input device for receiving a tonometer reading from a separate tonometer;
said microprocessor configured to execute at least one algorithm for modifying said tonometer reading based on the corneal thickness data to produce a corrected intraocular pressure value; and,
a display for displaying the corrected intraocular pressure value.

2. A device according to claim 1 wherein the pachymeter comprises
a pulse generator;
an ultrasonic transducer connected to generate an ultrasonic pulse in response to a pulse from the pulse generator; and,
a circuit for detecting reflected ultrasonic signals reflected at a back side of the cornea, amplifying the detected reflected ultrasonic signals and measuring a time between the generation of the ultrasonic pulse and the detection of the reflected ultrasonic signals.

3. A device according to claim 2 wherein the circuit comprises a timing circuit comprising:
a capacitor;
a charging circuit connected to charge the capacitor at a rate which decreases with time;
a trigger circuit connected to cause the charging circuit to commence charging the capacitor from an initial state of charge when the pulse is delivered by the pulse generator;
a digital to analog converter connected to measure a voltage across the capacitor as of a time at which the reflected signals are detected.

4. A device according to claim 3 wherein the charging circuit comprises an electrical resistance connecting the capacitor to a source of a constant voltage.

5. A device according to claim 2 wherein the microprocessor has access to a plurality of algorithms for IOP correction which may be selected by the operator.

6. A device according to claim 2 wherein the at least one algorithm generates a corrected IOP based upon at least corneal thickness and one or more tonometer reading values.

7. A device according to claim 4 wherein the at least one algorithm involves computing a linear combination of the corneal thickness and one or more tonometer reading values.

8. A device according to claim 7 wherein the at least one algorithm comprises a lookup table accessible to the microprocessor.

9. A device according to claim 1 comprising means for setting parameters of the at least one algorithm by way of the input device.

10. A device according to claim 1 comprising at least one stored set of a plurality of predetermined locations for taking a set of pachymeter measurements wherein the display is configured to display a graphical representation of an eye marked with indicia to graphically indicate one of the predetermined locations at which a next pachymeter measurement of the set ought to be taken.

11. A device according to claim 10 wherein upon obtaining the next pachymeter measurement the display is automatically configured to cause the indicia to graphically indicate another one of the predetermined locations.

12. A device for measuring thickness of a layer, the device comprising:
a transmitting transducer connected to transmit an ultrasonic pulse into a layer to be measured;
a receiving transducer connected to detect the ultrasonic pulse after the ultrasonic pulse has been reflected from a back side of the layer;
a timing circuit comprising:
a capacitor;
a charging circuit connected to charge the capacitor at a rate which decreases with time;
a trigger circuit connected to cause the charging circuit to commence charging the capacitor from an initial state of charge when an ultrasonic pulse is transmitted into the layer by the ultrasonic transducer;
a digital to analog converter connected to measure a voltage across the capacitor as of a time at which the reflected ultrasonic pulse is detected at the receiving transducer.

13. A device according to claim 12 wherein one transducer serves both as the receiving transducer and the transmitting transducer.

14. A pachymeter comprising a display and at least one stored set of a plurality of predetermined locations for taking a set of pachymeter measurements wherein the display is configured to display a graphical representation of an eye marked with indicia to graphically indicate one of the predetermined locations at which a next pachymeter measurement of the set ought to be taken.

15. A pachymeter according to claim 14 comprising means for automatically updating the display to cause the indicia to graphically indicate another one of the predetermined locations in response to a measurement being completed.
